(19) United States
(12) Patent Application Publication
(10) Pub. No.: US 2026/0272602 A1
(43) Pub. Date: Sep. 17, 2026

(54) INTRAOPERATIVE AND PHANTOM-LESS CALIBRATION OF REGISTRATION OF IMAGING SYSTEM WITH INTRAOPERATIVE IMAGING TO REFERENCE IMAGING

(71) Applicant: THE TRUSTEES OF DARTMOUTH COLLEGE, Hanover, NH (US)

(72) Inventors: Keith D. PAULSEN, Hanover, NH (US); William R. WARNER, Hanover, NH (US); Xiaoyao FAN, Hanover, NH (US)

(21) Appl. No.: 19/471,291

(22) PCT Filed: Apr. 25, 2024
(86) PCT No.: PCT/US2024/026271

§ 371 Date: Sep. 30, 2025

Related U.S. Application Data
(60) Provisional application No. 63/462,176, filed on Apr. 26, 2023.

(51) Int. Cl. A61B 90/00; G06T 7/33

(52) U.S. Cl. CPC A61B 90/37; G06T 7/337; A61B 2090/364; A61B 2090/378; G06T 2207/10064; G06T 2207/10081; G06T 2207/10088; G06T 2207/10136

(57) ABSTRACT

A method of registering intraoperative medical images to reference medical images includes: generating early-intraoperative medical images with an intraoperative medical imaging system; registering the reference medical images to the early-intraoperative medical images while computing a transform between coordinates of corresponding features in the reference medical images and the early-intraoperative medical images, the transform using location of the intraoperative medical imaging system as determined by a tracker; obtaining later intraoperative medical images; and applying the transform to register the reference medical images to the later intraoperative medical images. A system for generating and displaying reference and intraoperative medical images has a tracker attached to the intraoperative imaging machine and a registration machine configured to register the reference medical images to early-intraoperative medical images while recording a transform and to use that transform when registering later intraoperative medical images to the reference medical images.

### RELATED APPLICATIONS

The present document claims priority to U.S. provisional patent application 63/462,176 filed Apr. 26, 2023. The entire contents of the aforesaid provisional application are incorporated herein by reference.

### GOVERNMENT RIGHTS

This invention was made with government support under grant R01CA159324 awarded by the National Institutes of Health. The Government has certain rights in the invention.

### DEFINITIONS

Throughout this document, the following terms are used:

Reference imaging: Reference images are either a preoperative or very-early intraoperative image that would normally be used for surgical navigation. It is typically voxel-based such as a magnetic resonance imaging (MRI) or X-ray computed tomography (CT) image set. Since it is used for surgical navigation, it would be registered to the surgical field and has a registration matrix that can be used for calculation. Typically, this is a preoperative image set, but it in some cases be a very early intraoperative images and in some particular embodiments it may be intraoperative images in order to give an accurate calibration. Reference images may involve radiation exposure and often provide high resolution using contrast agents to best resolve tissues. In some embodiments, reference images have been annotated to flag tissues of particular interest such as critical structures or tissues (such as tumors) intended to be removed during surgery.

Early-intraoperative images: these are images of a patient prepped for surgery and taken by an intraoperative imaging system where a rigid tracker is attached to the system and patient. Intraoperative imaging systems may differ from those used for reference images because of radiation exposure hazards, contrast agent metabolism, imaging system bulk and immobility, and other factors; these images may have reduced contrast or resolution from reference images.

Later-intraoperative images: these are images that have been acquired by an intraoperative imaging system after a calibration has been acquired and may be acquired at various stages of surgery. In embodiments, these images are overlaid onto co-registered reference images to provide the physician with useful intraoperative information, such as current location of the tissues of particular interest of the reference images and are also useful for documenting surgical procedures. For example, later-intraoperative images may be of use to visualize remaining tumor that needs to be removed, or to visualize intraoperative locations of critical structures like nerves that surgeons wish to avoid damaging.

### BACKGROUND

Image guidance plays a significant role in surgical procedures.

Typically, reference medical images of the patient, such as MRI (magnetic resonance imaging), positron emissions tomography (PET scans), and CT (computed tomography), are acquired pre-operatively; in some cases, these images may be obtained as surgery begins. These images provide 3-dimensional maps of specific patients allowing surgeons to determine where critical structures and other tissue of interest were, how they related to other structures, diagnose issues such as tumors, and plan surgery; those reference images are, however, accurate and complete only as of the time those images were obtained.

Many tissues, typically including critical structures, other tissues of interest, and much surrounding tissue, are soft tissues that deform from the time of reference images as patients are positioned for surgery, as surgery proceeds and pressures on tissues change, and as tissue is displaced by surgical instruments, thus intraoperative positions of critical structures and other tissues of interest can differ significantly from their positions shown in reference images. Intraoperative imaging of patients can show interoperative positions, but often lack needed detail.

Some systems have been developed that register reference images to intraoperative images to show better detail of these critical structures and other tissues of interest to provide improved navigation information and guide surgery.

In some surgical procedures, including many neurosurgical procedures, trackers are attached to surgical instruments and instrument positions are registered to the reference images, this can aid surgeons in intraoperative guidance of those instruments by showing where they are in a 3-dimensional map of specific patient anatomy.

Not only can surgical instruments be tracked, and their location visualized onto these co-registered maps, but other imaging modalities can be tracked and coregistered, such as 2D and 3D ultrasound (US), 3-D laser scans, 3-D stereovision images, fluorescent images, or intraoperative X-ray such as fluoroscopic images, or CT images. This is particularly useful because patient anatomy often changes during surgery as soft tissues swell or are deformed, portions are removed, or implants inserted. These changes can be visualized using later-intraoperative images; co-registered later-intraoperative images are of use to guide further surgery. In some systems, it may be desirable to predict tissue stress or deformation resulting from insertion of implants.

In some surgeries where intraoperative US or CT is desired to be registered to preoperative images, a 2D or 3D ultrasound (US) probe of an ultrasound imaging machine is rigidly coupled with a tracking apparatus that in turn is tracked by a tracking device installed in the operating room. By doing so, the US images can be mapped to preoperative CT or MRI imaging coordinates allowing for merging of the two imaging modalities. This can provide a surgeon with real-time imaging from US or other intraoperative imaging that is overlaid onto preoperative imaging to allow visualization of intraoperative conditions in the patient.

Each imaging modality typically operates according to its own coordinate system. To properly map images obtained in a first modality such as ultrasound 102 (FIG. 1) using coordinate system F_{US} to a coordinate system Fₜᵣ of a tracker base station 104, to intraoperative patient position 106 in coordinate system Fₚₜ or to images obtained in another modality such as preoperative CT or MRI 108 using coordinate system F_{MR} typically requires a calibration that denotes how coordinates F_{US} in the first modality relate to the attached tracker (rotation and translation transform ^{tr}T_{sc}), how coordinates F_{MR} in the second modality relate to the attached tracker, and how coordinates according to the attached tracker relate to the patient's coordinates Fₚₜ. To perform this calibration typically requires imaging a phantom of known geometry to solve for a linear or nonlinear set of equations to calculate the transformations.

In traditional systems, each preoperative and intraoperative imaging system typically requires its own calibration prior to surgery—usually performed once for multiple surgeries; the accuracy of this calibration often must be verified at the start of each surgery.

### SUMMARY

In an embodiment, a method of registering intraoperative medical images to reference medical images includes: generating early-intraoperative medical images with an intraoperative medical imaging system; registering the reference medical images to the early-intraoperative medical images while computing a transform between coordinates of corresponding features in the reference medical images and the early-intraoperative medical images, the transform using location of the intraoperative medical imaging system as determined by a tracker; obtaining later intraoperative medical images; and applying the transform to register the reference medical images to the later intraoperative medical images.

In another embodiment, a system for generating and displaying reference and intraoperative medical images has a tracker attached to the intraoperative imaging machine and a registration machine configured to register the reference medical images to early-intraoperative medical images while recording a transform and to use that transform when registering later intraoperative medical images to the reference medical images.

### BRIEF DESCRIPTION OF THE FIGURES

FIG. 1 is a PRIOR ART illustration of the various coordinate systems that may be used for ultrasound, operating room tracking, and magnetic resonance imaging MRI or computed X-Ray tomography CT imaging, together with transformations typically required.

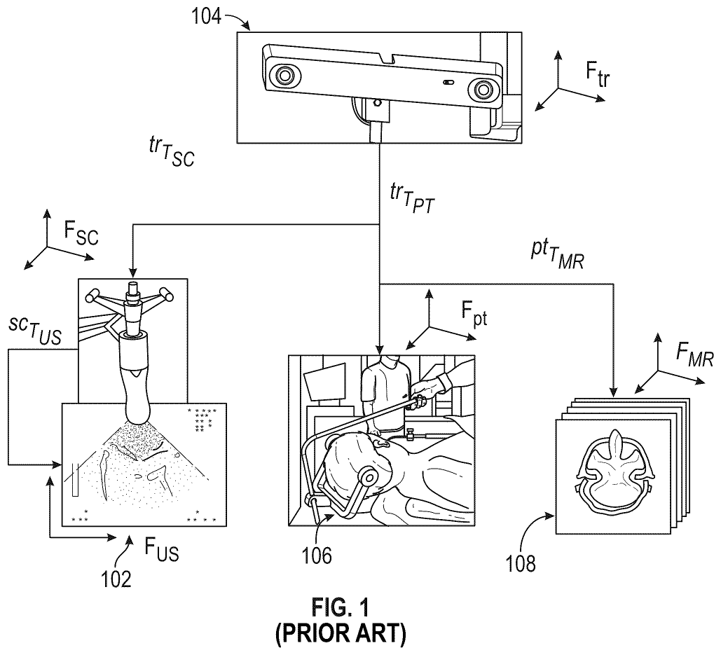

FIG. 2 is an illustration of the transformations required to map coordinate systems in a surgical system.

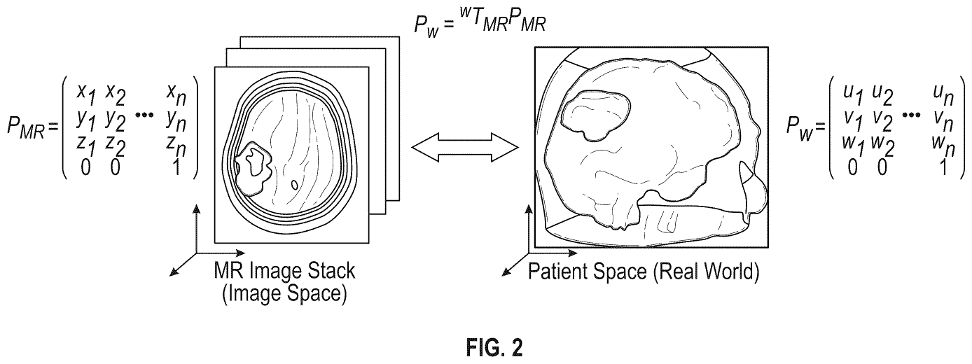

FIG. 3 is a block diagram of a surgical system.

FIG. 4 is a flowchart of a method of determining a calibration transform, in an embodiment.

### DETAILED DESCRIPTION OF THE EMBODIMENTS

In the following equations, ii: is intraoperative imaging, PRF: is patient reference frame, and Ri: is Reference imaging:

In a typical calibration flow:

Before surgery, a calibration is performed and calibration matrix ^{tracker}Tᵢᵢ is computed, and

At the start of surgery, patient registration is performed and a transformation matrix ^{Ri}T_{PRF} is computed to establish the relationship between the physical space (defined by the patient reference frame) and the reference imaging space.

To map a point from intraoperative imaging data (Pᵢᵢ) to the same point that is in reference imaging space (P_{Ri}), a series of transformations are performed

$${P}_{Ri} = \text{?} {T}_{PRF} {\times }^{PRF} {T}_{Localizer} \times {}^{Localizer} {T}_{tracker} \times {}^{tracker} {T}_{i i} \times {P}_{i i}$$

$$\text{?} \text{indicates text missing or illegible when filed}$$

Where the transformation from image point to reference image point can be combined into one transformation matrix:

$${}^{Ri} {T}_{ii} = \text{?} {T}_{PRF} \times {}^{PRF} {T}_{Localizer} \times {}^{Localizer} {T}_{tracker} \times {}^{tracker} {T}_{i i}$$

$$\text{?} \text{indicates text missing or illegible when filed}$$

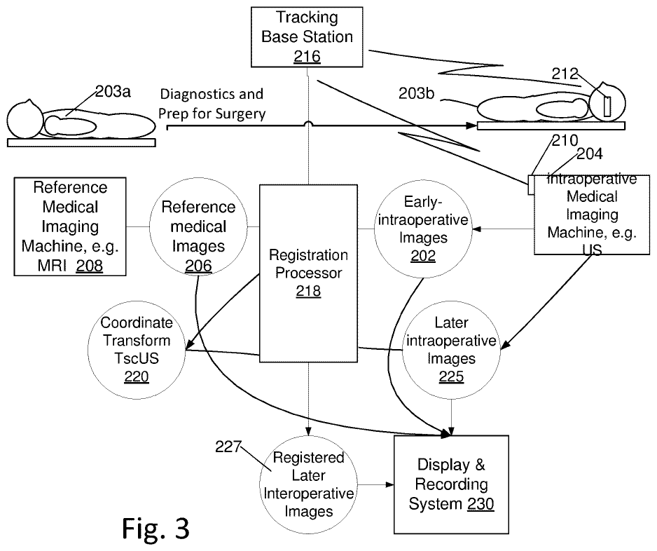

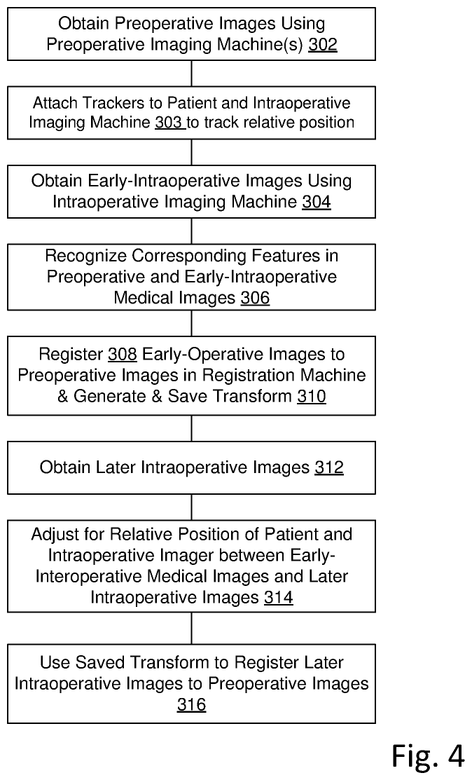

As disclosed herein, embodiments are discussed in terms of early-intraoperative images 202 (FIG. 3) and intraoperative images of a patient 203b, which are typically obtained 304 (FIG. 4) in the operating room just before, or during, a surgical procedure; the early-intraoperative images 202 being typically obtained of the patient 203b just before a surgical procedure or a first set of images taken during early phases of the surgery; with later intraoperative images being taken later during the surgery. These early and later intraoperative images may in embodiments be images obtained with ultrasound, stereovision, X-rays or X-ray computed tomography (CT) imaging machines 204. Discussion will also refer to reference medical images 206 of the reference patient 203a, which are typically obtained 302 prior to, and often days or weeks before, commencement of surgery and typically obtained using one or more imaging machines 208 such as magnetic resonance imaging (MRI) machines, gamma cameras, positron emissions tomography machines, ultrasound machines, and CT machines many of which may be used with and/or without contrast to best image tissues of interest. Typically, reference patient 203a and intraoperative patient 203b are the same patient but the intraoperative patient 203b may have been moved and repositioned from reference patient 203a positioning because intraoperative patient 203b has been positioned as needed for surgery. For example, the intraoperative patient may be positioned such that a surgeon can access in a reasonable way the organ or tissue requiring surgical intervention. The reference images are registered to the patient as positioned for surgery using either a fiducial-based or a fiducial-less method. In some embodiments, the reference images are obtained after the patient is positioned for surgery using a first imaging method, such as a portable CT machine, and early intraoperative and later intraoperative images are obtained with a second imaging method such as ultrasound, stereovision, fluorescence imaging, or other modalities. The disclosed intraoperative imaging system may be calibrated by registering the reference medical images 206 and the early-intraoperative images 202, using either a fiducial based or fiducial-less approach; in both fiducial and fiducial-less approaches corresponding features of the images are identified. A first tracker 210 is rigidly attached 303 to an imaging machine 204 suitable for making intraoperative images such as an ultrasound probe if ultrasound intraoperative images are to be used, a stereovision camera system if intraoperative stereovision images are to be used, or an X-ray or CT machine if intraoperative X-ray or CT images are to be used. In many embodiments, a second tracker 212 is attached to the patient 203b once the surgical field is prepared. A tracking base unit 216 tracks position and angle of the first tracker 210 and second tracker 212 and feeds these locations and angles to a registration machine 218 so any difference in relative positions of patient and intraoperative imaging machine between sets of early-intraoperative and later intraoperative images can be tracked and compensated for.

Early-intraoperative images are the images used to find a calibration between the images of this intraoperative imaging system and the tracker attached to this system. In most embodiments, the images acquired in this early state should match as closely as possible to the patient's state in the reference images, for example if preoperative MRI images are reference images for brain surgery and ultrasound for intraoperative imaging, the early-intraoperative ultrasound should be acquired before dural opening to minimize any brain shift between the MRI and ultrasound images.

The imaging machine 204 for making intraoperative images is used to make an initial early-intraoperative image of the patient, at this point both reference medical images 206 and early-intraoperative medical images 202 have been acquired and those images, together with tracked locations, are brought into the registration machine 218, the registration machine incorporates a processor and memory with machine readable instructions configured to perform registration.

The tracker may also be used while making intraoperative images. For example, of making an initial early-intraoperative image of the patient, when using a two dimensional (2D) ultrasound (US) probe, the probe can be used to make a volumetric sweep of anatomy of interest such as a volumetric sweep of brain before dural opening but after a craniotomy window has been made through the skull. This volumetric sweep with a tracked 2D US probe can be used to reconstruct a three-dimensional (3D) early-intraoperative imaging volume of the anatomy of interest. Alternatively, a 3D US probe can be used to directly acquire a volume of the anatomy of interest. Similarly, later-intraoperative images may also be obtained by doing a tracked volumetric sweep of a 2D US probe and constructing a 3D intraoperative imaging volume or by using a 3D US probe to directly acquire a volume of the anatomy of interest.

Once the reference and early-intraoperative images exist, corresponding features are identified 306 in both sets of images; in some embodiments these corresponding features include one or more fiducials, in other embodiments they include features of the patient visible in both sets of images such as bony landmarks.

Knowing the relative location in patient anatomy of where the US sweep was performed or where other images obtained, an image-based registration 308 is performed between the early-intraoperative images and the reference images, in a particular embodiment by using Mutual Information (MI) algorithms in a registration processor. In an alternative embodiment, an iterative closest point (ICP) algorithm is used in the registration processor to align surface data extracted from the reference and early intraoperative images. This registration between the reference and early intraoperative images involves determining 310 a transform between coordinates of the corresponding features identified in the reference and early-intraoperative medical images, this transform is used to calculate a calibration transform 220 given by TscUS below. Essentially, using the alignment between the reference images (e.g. MRI) and the early-intraoperative images (e.g. US), and by concatenating the spatial transformation to compute reference to early intraoperative space, we solve for an accurate calibration transformation between the reference medical images, the early-intraoperative images, and a location of a tracker 210 that is rigidly attached to the intraoperative imaging machine 204.

In particular embodiments, the reference medical images are MRI images, and the intraoperative images are ultrasound images. In other particular embodiments, the reference medical images are CT images, and the intraoperative images are ultrasound images, stereovision images, fluorescent imaging, or fluoroscopic images.

In some embodiments, a tracker 212 is also attached to the patient, this permits determining any differences in relative position between the patient and intraoperative medical imager during obtaining of the early-intraoperative medical images versus the patient and intraoperative medical imager during obtaining of later intraoperative medical images 225. The calibration transform 220 is then compensated for these differences in relative position.

Image-based registration 308 of the reference medical images and early-intraoperative medical images is performed at the start of surgery, and a registration matrix ^{Ri}T_{PRF} is computed.
Define:

$${}^{Ri} {T}_{tracker} = {}^{Ri} {T}_{PRF} \times {}^{PRF} {T}_{Localizer} \times {}^{Localizer} {T}_{tracker}$$

Then Determine:

$$in \text{?} {(}^{Ri} {T}_{tracker} ) {\times }^{Ri} {T}_{ii} = in \text{?} {(}^{Ri} {T}_{tracker} ) \times {}^{Ri} {T}_{tracker} {\times }^{tracker} {T}_{ii}$$

$$in \text{?} {(}^{Ri} {T}_{tracker} ) \times {}^{Ri} {T}_{ii} = {}^{tracker} {T}_{ii}$$

$$\text{?} \text{indicates text missing or illegible when filed}$$

Where ^{tracker}Tᵢᵢ is the calibration matrix to be solved for:
Therefore,

$${tracker}^{} {T}_{i i} = in \text{?} ( {}^{Ri} {T}_{PRF} \times {}^{PRF} {T}_{Localizer} \times {}^{Localizer} {T}_{tracker} ) \times {}^{Ri} {T}_{i i}$$

$$\text{?} \text{indicates text missing or illegible when filed}$$

where
   ^{Ri}Tᵢᵢ is solved via various embodiments
   ^{Ri}T_{PRF} is solved via accurate patient registration
   ^{PRF}T_{Localizer} and ^{Localizer}T_{tracker} are acquired via a tracking system

This gives a transformation between the reference images Ri to the patient reference frame PRF and to the early-intraoperative images ii.

In performing the registration, an Image Based Registration, such as a Mean Squared Difference registration, or a Mutual Information registration may be used. Such registration may take advantage of fiducials or features, such as bony landmarks, that are present in both the reference and in the early-intraoperative images. In alternative embodiments, a feature extraction is performed on both the reference and early-intraoperative images, and a Point Cloud Based Registration is performed which may use an Iterative closest point, Coherent Drift, or a Deep learning-based registration approach.

When later intraoperative medical images are obtained, the saved coordinate transform 220 is adjusted 314 for tracked differences in relative position between patient 203b and intraoperative imaging machine 204, and then used by the registration machine 218 to aid registration. The registration machine thus rapidly maps 316 the reference medical images 206 to the later-intraoperative medical images 225. Once the reference medical images are registered to the later-interoperative medical images, the later-interoperative medical images 225 may be further updated to show presence of tracked surgical tools or other hardware. Any combination of reference medical images 206, early-intraoperative medical images 202, later-interoperative medical images 225, and registered medical images 227 may then be displayed on a display system 230 and recorded as part of the patient's medical records.

Image Modality Registration Embodiments

| Intraoperative Imaging Data | Reference Images |
|---|---|
| Volume: | Volume |
| single volume | |
| multiple volumes combined | |
| tracked or untracked volume | |
| reconstruction method (2D US) | |
| Stitched volumes to create a composite | |
| volume | |
| Surface | Surface |
| single surface | |
| multiple surfaces combined | |
| stitched images to create a composite | |
| surface | |

Volume = image data represented in a volumetric voxel grid
Surface = point cloud with an optional texture map - this can be extracted from a volume in the case of MRI or CT images

In embodiments, the intraoperative images are selected from the group: intraoperative stereovision, structured light microscopy, fluorescence imaging, optical scanners, fluoroscopy, 3D ultrasound, 2D ultrasound, portable X-ray, portable CT, and others.

### Combinations of Features

The various features herein described can be combined in various ways. Among combinations anticipated by the inventors are:

A method designated A of registering intraoperative medical images to reference medical images including: generating early-intraoperative medical images with an intraoperative medical imaging system; registering the reference medical images to the early-intraoperative medical images while computing a transform coordinates of corresponding features in the reference medical images and the early-intraoperative medical images, the transform using location of the intraoperative medical imaging system as determined using a tracker; obtaining later intraoperative medical images; and applying the transform between coordinates of corresponding features in the reference medical images and the early-intraoperative medical images to register the reference medical images to the later intraoperative medical images.

A method designated AA including the method designated A where the reference medical images are selected from magnetic resonance imaging (MRI) medical images and X-ray computed tomography (CT) medical images, and the early-intraoperative and later intraoperative medical images are selected from stereovision, fluoroscopy, ultrasound and fluorescence images.

A method designated AB including the method designated A or AA where the early-intraoperative and later intraoperative medical images are selected from three-dimensional and a plurality of two-dimensional ultrasound images.

A method designated AC including the method designated A, AA, or AB further includes using the tracker to track changes in relative position between the intraoperative medical imaging system and adjusting the transform according to the changes in relative position of the intraoperative medical imaging system.

A system designated B for generating and displaying reference and intraoperative medical images includes: a first tracker attached to an intraoperative imaging machine; a tracker base station coupled to provide tracked locations of the first tracker to a registration machine; the registration machine configured to register reference medical images to early-intraoperative medical images obtained with the intraoperative imaging machine while recording a transform between coordinates of corresponding features in the reference medical images and the early-intraoperative medical images, and to use that transform when registering later intraoperative medical images to the reference medical images.

A system designated BA including the system designated B wherein the transform further considers coordinates of the first tracker.

A system designated BB including the system designated B or BA further comprising a second tracker attached to a patient.

A system designated BC including the system designated B, BA, or BB where the intraoperative imaging machine comprises a probe of an ultrasound imaging machine.

A system designated BD including the system designated B, BA, or BB where the intraoperative imaging machine is selected from the group consisting of fluoroscopy machines, fluorescent imaging machines, intraoperative CT machines, laser scanners, and stereovision imagers.

A method designated C of registering intraoperative medical images to reference medical images includes: generating early-intraoperative medical images with an intraoperative medical imaging system; registering the reference medical images to the early-intraoperative medical images while computing a transform between coordinates of corresponding features in the reference medical images and the early-intraoperative medical images, the transform using a location of the intraoperative medical imaging system determined with a tracker; obtaining later intraoperative medical images; and applying the transform between coordinates of corresponding features in the reference medical images and the early-intraoperative medical images to register the reference medical images to the later intraoperative medical images.

A method of registering intraoperative medical images to reference medical images designated CA including the method designated C where the reference medical images are selected from magnetic resonance imaging (MRI) medical images and X-ray computed tomography (CT) medical images, and the early-intraoperative and later intraoperative medical images are selected from stereovision, fluoroscopy, ultrasound and fluorescence images.

A method of registering intraoperative medical images to reference medical images designated CB including the method designated CA or C where the early-intraoperative and later intraoperative medical images are selected from three-dimensional and a plurality of two-dimensional ultrasound images.

A method of registering intraoperative medical images to reference medical images designated CC including the method designated C, CA, or CB further comprising using the tracker attached to the intraoperative medical imaging system to track changes in relative position of the intraoperative medical imaging system, and adjusting the transform according to the changes in relative position of the intraoperative medical imaging system between the early-intraoperative medical images and the later-intraoperative medical images.

A method of registering intraoperative medical images to reference medical images designated CD including the method designated C, CA, CB, or CC further comprising using a second tracker to track position of a patient and adjusting the transform according to the changes in relative position of the patient between the early-intraoperative medical images and the later-intraoperative medical images.

Changes may be made in the above system, methods or device without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

1. A method of registering intraoperative medical images to reference medical images comprising:
generating the reference medical images with a medical imaging system;
generating early-intraoperative medical images with an intraoperative medical imaging system;
registering the reference medical images to the early-intraoperative medical images while computing a transform coordinates of corresponding features in the reference medical images and the early-intraoperative medical images, the transform using location of the intraoperative medical imaging system as determined using a tracker;
obtaining later intraoperative medical images; and
applying the transform between coordinates of corresponding features in the reference medical images and the early-intraoperative medical images to register the reference medical images to the later intraoperative medical images.

2. The method of claim 1 where the reference medical images are selected from magnetic resonance imaging (MRI) medical images and X-ray computed tomography (CT) medical images, and the early-intraoperative and later intraoperative medical images are selected from stereovision, fluoroscopy, ultrasound and fluorescence images.

3. The method of claim 2 where the early-intraoperative and later intraoperative medical images are selected from three-dimensional and a plurality of two-dimensional ultrasound images.

4. The method of claim 1, further comprising using the tracker to track changes in relative position between the intraoperative medical imaging system and adjusting the transform according to the changes in relative position of the intraoperative medical imaging system.

5. A system for generating and displaying reference and intraoperative medical images comprising:
a first tracker attached to an intraoperative imaging machine;
a tracker base station coupled to provide tracked locations of the first tracker to a registration machine;
the registration machine being configured to register reference medical images to early-intraoperative medical images obtained with the intraoperative imaging machine while recording a transform between coordinates of corresponding features in the reference medical images and the early-intraoperative medical images, and to use that transform when registering later intraoperative medical images to the reference medical images.

6. The system of claim 5 wherein the transform further considers coordinates of the first tracker.

7. The system of claim 6 further comprising a second tracker attached to a patient.

8. The system of claim 6 where the intraoperative imaging machine comprises a probe of an ultrasound imaging machine.

9. The system of claim 6 where the intraoperative imaging machine is selected from the group consisting of fluoroscopy machines, fluorescent imaging machines, intraoperative CT machines, laser scanners, and stereovision imagers.

10. A method of registering intraoperative medical images to reference medical images comprising:
generating early-intraoperative medical images with an intraoperative medical imaging system;
registering the reference medical images to the early-intraoperative medical images while computing a transform between coordinates of corresponding features in the reference medical images and the early-intraoperative medical images, the transform using a location of the intraoperative medical imaging system determined with a tracker;
obtaining later-intraoperative medical images; and
applying the transform between coordinates of corresponding features in the reference medical images and the early-intraoperative medical images to register the reference medical images to the later-intraoperative medical images.

11. The method of claim 10 where the reference medical images are selected from magnetic resonance imaging (MRI) medical images and X-ray computed tomography (CT) medical images, and the early-intraoperative and the later-intraoperative medical images are selected from stereovision, fluoroscopy, ultrasound and fluorescence images.

12. The method of claim 11 where the early-intraoperative and the later-intraoperative medical images are selected from three-dimensional and a plurality of two-dimensional ultrasound images.

13. The method of claim 10, further comprising using the tracker attached to the intraoperative medical imaging system to track changes in relative position of the intraoperative medical imaging system, and adjusting the transform according to the changes in relative position of the intraoperative medical imaging system between the early-intraoperative medical images and the later-intraoperative medical images.

14. The method of claim 13 further comprising using a second tracker to track position of a patient and adjusting the transform according to the changes in relative position of the patient between the early-intraoperative medical images and the later-intraoperative medical images.
